(19) United States
(12) Patent Application Publication
(10) Pub. No.: US 2025/0389707 A1
(43) Pub. Date: Dec. 25, 2025

(54) SYSTEMS AND METHODS FOR ESTIMATING SCALING IN A WATER FILTRATION SYSTEM

(71) Applicant: TexOPCO, LLC, Houston, TX (US)

(72) Inventors: Mavis C. Y. Wong, Houston, TX (US); Sunil Mehta, Henrico, VA (US); Chad Unrau, Houston, TX (US)

(21) Appl. No.: 19/245,134

(22) Filed: Jun. 20, 2025

Related U.S. Application Data
(60) Provisional application No. 63/662,585, filed on Jun. 21, 2024.

(51) Int. Cl. G01N 33/18

(52) U.S. Cl. CPC G01N 33/18

(57) ABSTRACT

Provided herein are systems and methods that estimate the amount of scaling in water filtration systems by determining scaling potential of the water fed to the water filtration system. The systems and methods described herein may receive parameters of the feed water and ion concentration data, and based on the ions present in the feed water and the ion concentration data, may determine one or more potential precipitates in the feed water. Based on the potential precipitates and the feed water parameters, the systems and methods described herein may determine a scaling potential of the feed water, which can be used to estimate the amount scaling that could be formed in a water filtration system. The systems and methods described herein may display output information comprising a representation of the determined scaling potential.

### PRIORITY

This application is a non-provisional conversion of and claims priority under 35 USC 119 from Provisional Application 63/662,585 filed Jun. 21, 2025 and entitled “SYSTEMS AND METHODS FOR ESTIMATING SCALING OF A MEMBRANE IN A WATER FILTRATION SYSTEM”, the entire contents of which are hereby incorporated by reference.

### FIELD

This disclosure relates generally to systems and methods for estimating scaling of membranes in a water filtration system, and more specifically to systems and methods for determine the scaling potential of a feed water supply to estimate the amount of scaling in the water filtration system.

### BACKGROUND

Membrane processes for desalination and water treatment are becoming increasingly popular in water intensive industries. Some of the benefits membrane processes provide include a reduction in the volume of waste, recycling and reuse of key ions or chemicals, and a small footprint. Some of the most common membrane processes include reverse osmosis (RO), nanofiltration (NF), ultrafiltration (UF), and electrodialysis (ED). While many benefits are associated with membrane processes, challenges are also present, including high energy consumption, production of highly concentrated waste, requirement of chemicals, and potentially high initial and maintenance costs. However, perhaps the largest challenge is the formation of scale on the membrane surface, which reduces the effective area of the membrane, creates a pressure drop, reduces flow, and can lead to irreversible damage to the membrane.

Some commercial models exist that attempt to predict the amount of scaling that may occur during water treatment based on the chemistry of the feed water. The primary method of predicting scaling is using the Langelier Saturation Index (LSI), which is an approximate indicator of the degree of saturation of calcium carbonate (CaCO₃). A negative LSI indicates that calcium carbonate is undersaturated, which leads to corrosion of the calcium carbonate coating in pipelines of the water filtration system. A positive LSI indicates that the water is supersaturated, and scale can form on the membrane. An LSI of 0 indicates water that is at saturation, so no calcium carbonate coating is removed and no scale forms. The LSI is used often because calcium carbonate is one of the most common, if not the most common, constituent of scale. However, the LSI is not a true indicator of scaling potential because many other constituents are present in scale, such as other calcium compounds, sulfates, phosphates, and hydroxides. The LSI does not account for these constituents, and often water with a desirable LSI will have considerable scaling. Additionally, many of these commercial models are made with reverse osmosis (RO) in mind and are not tailored to electrodialysis.

### SUMMARY

Disclosed herein are systems and methods that estimate the scaling potential of water in water filtration systems. Membranes do not exhibit scaling potential. The systems and methods described herein consider characteristics of the feed water and ions identified in the water to determine potential precipitates in the water, and based on the precipitates and feed water characteristics, determine the scaling potential of water in the filtration system. For example, aside from calcium carbonate precipitates, the systems and methods described herein may consider hydroxides, phosphates, and/or sulfate precipitates to estimate the scaling potential of the water. The systems and methods disclosed herein may improve upon existing scaling estimation tools and models by considering several parameters beyond just the Langelier Saturation Index (LSI) of the feed water. For example, the systems and methods may consider the relationship between pH, CO₂, and alkalinity to estimate the scaling potential. The systems and methods described herein may also consider the induction time in determining the scaling potential of the water. Moreover, the systems and methods described herein may be applicable in estimating the scaling potential of water filtration systems that include various types of membrane processes, including electrodialysis (ED), reverse osmosis (RO), nanofiltration (NF), and ultrafiltration (UF).

More specifically, the present disclosure provides a method for estimating the amount scaling in a water filtration system, comprising:
receiving feed water data comprising at least an identification of ions present in the feed water, concentration data for each of the ions present in the feed water, and a plurality of parameters of the feed water;
determining one or more potential precipitates in the feed water based on the identification of ions present in the feed water and the concentration data for each of the ions present in the feed water; and
based at least one the one or more potential precipitates and the plurality of parameters of the feed water, determining the scaling potential of the feed water to estimate the amount of scaling in the water filtration system.

Here is described that a plurality of parameters of the feed water comprises temperature, pH, CO₂ content, and/or alkalinity of the feed water.

It is also possible to determine the one or more potential precipitates is based on a solubility constant (Kₛₚ) and a product constant (Q) of one or more compounds formed by the identified ions in the feed water.

In this case it is possible to determine the one or more potential precipitates and comparing the solubility constant to the product constant for each of the one or more compounds in the feed water.

In another embodiment determining the one or more potential precipitates is based on an induction time of each of the one or more compounds formed by the identified ions in the feed water.

It is also important to determine the one or more potential precipitates by using a regression model that is configured to receive as input concentration data for each of said ions present in said feed water, solubility constant data, product constant data, and/or feed water pH data, and to provide as output whether precipitates will form and if one or more potential precipitates will precipitate out of solution.

Here the one or more potential precipitates comprises calcium, carbonate, hydroxide, phosphate, and/or sulfate precipitates.

This method also determines the scaling potential of the membrane comprises comparing a determined Langelier Saturation Index (LSI) of the feed water to a predetermined threshold range wherein said scaling potential utilizes a model that reviews all common scaling compounds and feed water composition to determine if ions are present in said feed water wherein an absence of ions in said scaling compound leads to no precipitate and wherein a product constant (Q) is calculated and compared it a solubility constant (Ksp) of said product such that if Q>Ksp there is potential for scaling to occur and wherein pH and calcium, carbonate content determines said Langelier Saturation Index (LSI) and if LSI>1, said LSI and said Ksp and said ion concentration determine a degree of scaling and wherein if LSI<1, said Ksp and ion concentration are combined to determine a degree of scaling.

In addition this method allows for displaying output information to mitigate membrane scaling based on the determined scaling potential of the water wherein the displayed output information comprises a representation of the scaling potential and wherein the representation of the scaling potential comprises a severity scale.

The displayed output information comprises the Langelier Saturation Index (LSI) of the feed water and wherein the displayed output information comprises a representation of the ions present in the feed water and/or a representation of the one or more potential precipitates in the feed water.

In further embodiments the output information comprises one or more anti-scalant solutions to be added to the water filtration system, a dosage of the one or more anti-scalant solutions to be added to the water filtration system, and/or a flow rate for adding the one or more anti-scalant solutions to the water filtration system.

Here, the membrane in the water filtration system utilizes electrodialysis, reverse osmosis, nanofiltration, and/or ultrafiltration.

The present disclosure also provides for a system for estimating scaling of a membrane in a water filtration system, the system comprising one or more processors configured to cause the system to:
receive feed water data comprising at least an identification of ions present in the feed water, concentration data for each of the ions present in the feed water, and a plurality of parameters of the feed water;
determine one or more potential precipitates in the feed water based on the identification of ions present in the feed water and the concentration data for each of the ions present in the feed water; and
based at least one the one or more potential precipitates and the plurality of parameters of the feed water, determine a scaling potential of the feed water to estimate scaling in the water filtration system.

The system utilizes a plurality of parameters of the feed water comprises temperature, pH, CO2 content, and/or alkalinity of the feed water and for determining the one or more potential precipitates is based on a solubility constant (Kₛₚ) and a product constant (Q) of one or more compounds formed by the identified ions in the feed water.

Here the system is useful in determining the one or more potential precipitates comprises comparing the solubility constant to the product constant for each of the one or more compounds in the feed water.

In another embodiment the system determines the one or more potential precipitates based on an induction time of each of the one or more compounds formed by the identified ions in the feed water.

Further the system is determining the one or more potential precipitates that comprises using a regression model configured to receive as input the concentration data for each of the ions present in the feed water, solubility constant data, product constant data, and/or feed water pH data, and to provide as output the one or more potential precipitates wherein the one or more potential precipitates comprises calcium, carbonate, hydroxide, phosphate, and/or sulfate precipitates.

The system also allows for determining the scaling potential of the membrane comprises comparing a determined Langelier Saturation Index (LSI) of the feed water to a predetermined threshold range and further comprises displaying output information to mitigate membrane scaling based on the determined scaling potential of the water.

Here the displayed output information comprises a representation of the scaling potential and the representation of the scaling potential comprises a severity scale wherein the displayed output information comprises the Langelier Saturation Index (LSI) of the feed water.

In addition, the system displays output information that comprises a representation of the ions present in the feed water and/or a representation of the one or more potential precipitates in the feed water.

The system also provides output information that comprises one or more anti-scalant solutions to be added to the water filtration system, a dosage of the one or more anti-scalant solutions to be added to the water filtration system, and/or a flow rate for adding the one or more anti-scalant solutions to the water filtration system, wherein the membrane in the water filtration system utilizes electrodialysis, reverse osmosis, nanofiltration, and/or ultrafiltration.

In a further embodiment, the present disclosure provides for a non-transitory computer-readable storage medium storing one or more programs for estimating the amount of scaling in a water filtration system, the programs for execution by one or more processors of an electronic device that when executed by the device, cause the device to:
receive feed water data comprising at least an identification of ions present in the feed water, concentration data for each of the ions present in the feed water, and a plurality of parameters of the feed water;
determine one or more potential precipitates in the feed water based on the identification of ions present in the feed water and the concentration data for each of the ions present in the feed water; and
based at least one the one or more potential precipitates and the plurality of parameters of the feed water, determine a scaling potential of the feed water to estimate the amount of scaling in the water filtration system.

In some embodiments, any one or more of the parameters of any one or more of the systems, methods, and/or computer-readable storage mediums recited above may be combined, in whole or in part, with one another and/or with any other features or parameters described elsewhere herein.

### BRIEF DESCRIPTION OF THE FIGURES

Various aspects of the disclosed systems and methods are set forth with particularity in the appended claims. A better understanding of the features and advantages of the disclosed systems and methods will be obtained by reference to the detailed description of illustrative embodiments and the accompanying drawings.

FIG. 1 shows a system for estimating the scaling of a membrane in a water filtration system, in accordance with some aspects.

FIG. 2 shows a method for estimating the scaling of a membrane in a water filtration system, in accordance with some aspects.

FIG. 3 shows an example graphical user interface of the system for estimating scaling of the membrane in the water filtration system.

FIG. 4 shows a computer, in accordance with some embodiments.

### DETAILED DESCRIPTION

Systems and methods for estimating the amount of scaling in water filtration systems are described herein. The systems and methods described herein may consider characteristics of the feed water and ions identified in the feed water to determine potential precipitates in the feed water. Based on the determined potential precipitates and one or more characteristics of the water in the water filtration system, the systems and methods described herein may determine the scaling potential of the feed water. The systems and methods described herein may be configured to display output information related to the scaling potential of the water, such as a severity scale and/or textual representation of the determined scaling potential. In some instances, the systems and methods described herein may generate and display a recommendation to mitigate the scaling in the water filtration system.

The systems and methods described herein may improve upon existing systems and methods for estimating scaling because those described herein may wholistically consider all precipitates that can be formed from the feed water, beyond calcium carbonates, to determine the scaling potential. Also, the systems and methods described herein may consider various parameters of the feed water and of the ions present in the feed water to determine the scaling potential of the water. For example, the systems and methods may consider the relationship between pH, CO₂, and total alkalinity, as well as induction time of precipitates within the feed water, each of which heretofore may not have been considered in estimating scaling. Accordingly, the systems and methods provided herein may achieve a more accurate estimation of scaling in the water filtration system. Also, the systems and methods provided herein may provide a clearer indication of how to interpret and act on the determined scaling potential to improve mitigation efforts of scaling in the water filtration system. Further, the methods and systems described herein may be utilized to estimate scaling potential of feed streams, brine streams, product streams, and water within the water filtration system, one or more of which heretofore may not have been considered in estimating the scaling potential of water in the filtration system.

The disclosure will be described first with respect to an exemplary system for estimating scaling of a water filtration system, followed by methods for execution by the system for estimating the scaling in the filtration system. An example graphical user interface of the system configured to receive inputs and provide outputs to/from a user and/or a water filtration system is also provided herein. Finally, an example computing device that can comprise one or more aspects of the system for estimating scaling of a membrane of a water filtration system will be described herein.

### Systems for Estimating Scaling in Water Filtration Systems

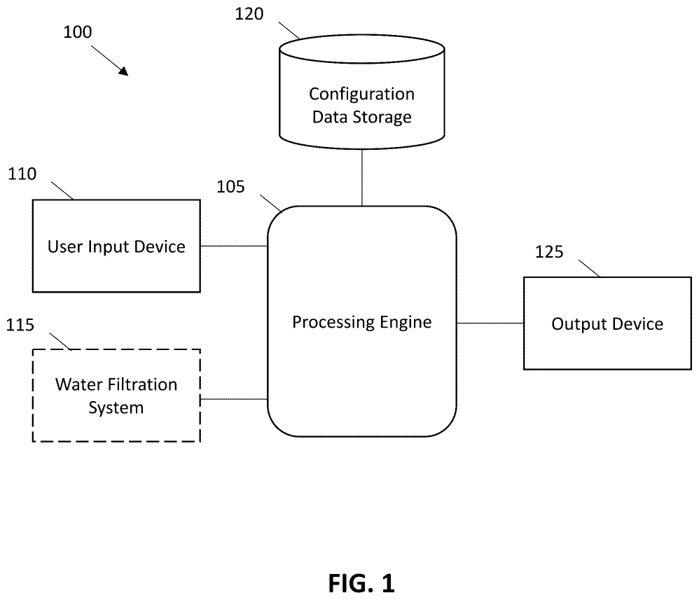

FIG. 1 depicts a system 100 for estimating the scaling in a water filtration system, in accordance with some aspects.

System 100 may be a computerized system including one or more processors, one or more computer storage mediums, one or more communication devices, and one or more input/output devices. While the components of system 100 are shown, by way of example, in a particular arrangement in FIG. 1, a person of ordinary skill in the art will appreciate, in light of the disclosure herein, that one or more components of system 100 may be combined, provided by multiple separate systems, provided by a single system, and/or provided in a distributed arrangement. In some embodiments, one or more of the data processing functionalities of the various components of system 100 may be provided by a single processor, by a plurality of processors, and/or by a distributed processing system. In some embodiments, one or more of the data storage functionalities of the various components of system 100 may be provided by a single computer storage device (e.g., a single database or RAM), by a plurality of computer storage devices, and/or by a distributed computer storage system.

In the exemplary arrangement shown in FIG. 1, system 100 may include processing engine 105, a user input device 110, a water filtration system 115, configuration data storage 120, and an output device 125. Although each of the aforementioned components are illustrated as a single block in FIG. 1, it is to be understood that any one or more of the components may comprise one or more (e.g., a plurality) of the given component. For example, system 100 may comprise more than one input device, more than one data storage, more than one processing engine, more than one output device, etc. In some embodiments, system 100 may not explicitly comprise a water filtration system 115, signified by the dashed lines in the Figure. Rather, in some instances, data from the water filtration system 115 may be received at processing engine 105 via user input device 110, as will be described herein. Parameters of each of the aforementioned components of system 100 are described in greater detail below.

Processing engine 105 may comprise one or more computer processors configured to perform one or more of the data processing functionalities described herein. In some embodiments, processing engine 105 may be provided as a local processor or set of processors, and/or as a web-hosted processor or set of processors (e.g., distributed processors). In some embodiments, processing engine may include one or more central processing units (CPUs) and/or graphics processing units (GPUs).

The output generated by processing engine 105 may be stored by any suitable computer storage medium and in any suitable format, such as being provided as a part of one or more databases or RAM of processing engine 105. In some embodiments, the output generated by processing engine 102 may be stored in the configuration data storage 120 illustrated in FIG. 1. In some embodiments, the output of processing engine 105 may be stored in an external data storage or memory not explicitly illustrated in FIG. 1.

As shown in FIG. 1, a user input device 110 may be communicatively coupled (e.g., via one or more wired and/or wireless network communication interfaces) to processing engine 105 to transmit user inputs to the processing engine 105. The user input device 110 may comprise any one or more computers or computer systems, such as one or more personal computers, laptops, tablets, smart phones, mobile electronic devices, workstations, or the like comprising a keyboard, mouse, touch screen, microphone, graphical user interface, etc. to receive user inputs. In some embodiments, the system 100 may comprise a first user input device 110 (e.g., a back-end user input device) for configuring the processing engine 105, and/or a second user input device 110 (e.g., a front-end user input device) for providing inputs to the processing engine 105 for operation. In some embodiments, the front-end and back-end user input device may be the same device, for example, in the instance the intended front-end user and back-end user are the same user.

As a back-end input device, the user inputs may comprise configuration data for configuring operations of the processing engine 105. For example, configuration data may comprise an identification (e.g., a data structure such as a list, table, etc.) of various ions, compounds, precipitates, etc., that may be present in the water fed to the water filtration system and/or may form based on ions/compounds present in the feed water. In some embodiments, configuration data may comprise parameters related to the aforementioned ions and compounds, such as the name, chemical formula, solubility product (Kₛₚ), ion product (Q), and/or induction time of the ion/compounds. In some embodiments, the processing engine 105 may be communicatively coupled to one or more public or private resources comprising any amount of the aforementioned configuration data and may retrieve and store relevant information for estimating membrane scaling. In this instance, the user may not be required to provide as much, if any, configuration data to the processing engine 105 for operation. The configuration data, whether provided manually by a user, retrieved by the system from one or more online resources, or a combination thereof, may be stored for later operation by the processing engine 105, for example in configuration data storage 120.

As a front-end input device, the user inputs provided to the processing engine 105 via the user input device 110 may comprise feed water data. For example, a user may receive feed water data from the water filtration system 115 and provide the data to the processing engine 105 via the user input device 110. Alternatively, as described in greater detail below, the feed water data may be provided directly to the processing engine 105 from the water filtration system 115 (e.g., via one or more sensors of water filtration system 115 and/or a processor of the system 115) without substantive input from a user. The feed water data may comprise an identification of ions, ion concentrations in the feed water, and/or one or more parameters of the feed water, described in greater detail below.

As mentioned above, in some embodiments, one or more components of a water filtration system 115 may be communicatively coupled (e.g., via one or more wired and/or wireless network communication interfaces) to processing engine 105. Water filtration system 115 may utilize one or more filtration processes including but not limited to electrodialysis, reverse osmosis, nanofiltration, and/or ultrafiltration. Each of the aforementioned filtration processes may necessitate one or more membranes (e.g., filters) through which a water supply (e.g., feed water) may be passed. The water filtration system 115 may comprise one or more sensors configured to detect data related feed water streams in the filtration system, such as feed streams, product streams, brine streams, etc. The sensors may be directly or indirectly coupled to the processing engine 105. For example, the water filtration system 115 may comprise one or more processors communicatively coupled to the one or more sensors of water filtration system 115 to receive data from the one or more sensors and may be configured to automatically transmit the data to processing engine 105. In some embodiments, the one or more sensors may automatically (and directly) transmit feed water data to the processing engine 105.

It is contemplated that the water filtration system 115 may comprise one or more components of the system 100 described herein. For example, the water filtration system 115 may comprise the processing engine 105 (e.g., in addition to or in place of the aforementioned one or more processors of water filtration system 115, should they exist). In some embodiments, the water filtration system 115 may additionally or alternatively comprise one or more user input devices 110, output devices 125, and/or data storages (e.g., configuration data storage 120).

As will be described in greater detail below, processing engine 105 may be configured to operate on data (e.g., the feed water data) detected by sensors of the water filtration system 115 to estimate scaling in the water filtration system 115. For example, based on feed water data, scaling potential of the feed water stream, brine stream, product stream, and/or water within the filtration system may be estimated. The operations of the processing engine 105 may utilize, in addition to the feed water data, the above-described configuration data. The configuration data may be accessed by the processing engine 105 ad-hoc from one or more privately or publicly accessible resources, retrieved from an internal and/or external data storage (e.g., configuration data storage 120), and/or provided in real-time by a user via user input device 110.

As shown in FIG. 1 and mentioned above, the system 100 may comprise one or more data stores, such as configuration data storage 120. Configuration data storage 120 may be communicatively coupled (e.g., via one or more wired and/or wireless network communication interfaces) to processing engine 105. In some embodiments, one or more data stores (e.g., configuration data storage 120) may be embodied within components of system 100 (e.g., water filtration system 115, processing engine 105, etc.). The configuration data storage 120 may be periodically updated, for example, manually via actions of a user at user input device 110 (described above) and/or manually by processing engine 105. For example, data stores of system 100 (e.g., configuration data storage 120) may be configured to automatically update based on information provided to processing engine 105 by user input device 110, water filtration system 115, and/or output device 125. In some embodiments, configuration data storage 120 may be communicatively coupled to one or more privately or publicly accessible resources, and in turn may be configured to update based on information retrieved from the resources. System 100 may be configured such that some or all of the information stored in configuration data storage 120 may be communicated to processing engine 105 for processing as described herein. Namely, processing engine 105 may be configured to utilize or extract information from configuration data storage 120 to estimate scaling in water filtration system 115.

In some embodiments, system 100 may comprise one or more output devices 125. The output devices 125 may be communicatively coupled (e.g., via one or more wired and/or wireless network communication interfaces) to processing engine 105 to receive data from the processing engine 105. The output device 125 may include any one or more computers or computer systems, such as one or more personal computers, laptops, tablets, smart phones, mobile electronic devices, workstations, or the like comprising one or more displays configured to display information from processing engine 105. In some embodiments, the system 100 may comprise a first output device 125 (e.g., a back-end output device) for displaying processing outputs of the processing engine 105 (during configuration of the processing engine 105), and/or a second output device 125 (e.g., a front-end output device) for displaying outputs produced by processing engine 105. In some embodiments, one or more of the aforementioned output devices 125 may be provided in the same device as one or more of the aforementioned user input devices 110. For example, a single computer system (e.g., computer, laptop, tablet, smart phone, mobile electronic device, workstation, etc.) may function as a user input device and an output device, whether it be a front-end device or a back-end device. In some embodiments, the output device 125 may display a graphical user interface (GUI) configured to receive inputs from a user. In a non-limiting example, the output device 125 may display one or more outputs produced by processing engine 105, and the output device 125 may be configured to receive selections of a user on a GUI of the output device 125 comprising instructions to save one or more outputs of the processing engine 105 (e.g., in a data store, such as configuration data storage 120).

### Methods for Estimating Scaling in Water Filtration Systems

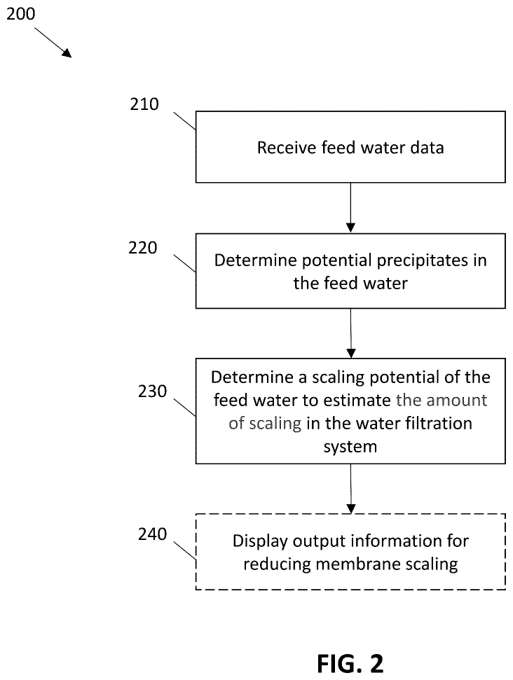

FIG. 2 illustrates an exemplary method 200 for estimating scaling of a membrane of a water filtration system, in accordance with some embodiments.

At block 210, the method 200 may comprise receiving feed water data comprising at least an identification of ions present in the feed water, concentration data for each of the ions present in the feed water, and a plurality of parameters of the feed water. The feed water data may be received from a water filtration system (e.g., water filtration system 115 described with respect to system 100 in FIG. 1) and/or a user (e.g., via user input device 110 in system 100 of FIG. 1). For simplicity, hereinafter, data received (e.g., by a processing engine of the system, such as processing engine 105 of system 100 illustrated in and described with respect to FIG. 1) is understood to encompass data received by a user (e.g., via a user input device) and data received by a water filtration system (e.g., via one or more sensors and/or processors of a water filtration system), unless explicitly stated otherwise.

In some embodiments, the processing engine may utilize a predetermined set of ions that may be present in feed water and may be configured to receive an indication of ions in the predetermined set that are present in a feed water supply. For example, configuration data storage 120 may store the set of ions in a structured data set that may be referenced by the processing engine. The processing engine may be configured to update the predetermined set of ions, for example, based on newly identified ions in the feed water, based on instructions provided via a user input device to update the predetermined set of ions to include one or more new ions, and/or based on information retrieved from one or more data resources. In some embodiments, rather than utilizing a predetermined set of ions to form a basis for ion identification by the processing engine, the processing engine may be configured to receive the identification of ions, and may then store the identified ions (e.g., temporarily in a RAM and/or in configuration data storage 120) for further processing.

In some embodiments, the predetermined set of ions that may be present in feed water may include but is not limited to hydrogen, hydroxide, aluminum, ammonium, barium, bicarbonate, boron, bromide, calcium, carbonate, chloride, fluoride, iron, magnesium, manganese, nitrate, phosphate, potassium, silica, sodium, strontium, and/or sulfate.

The processing engine may be configured to receive concentration data for each of the ions present in the feed water. The ion concentration data may be derived from one or more conductivity sensors of the water filtration system and transmitted (e.g., via the filtration system itself and/or by a user via a user input device) to the processing engine. Ion concentration data may be important in estimating the amount scaling in a water filtration system because, in combination with parameters of precipitates that may form from the ions present in the water (e.g., solubility constant, Kₛₚ, and product constant, Q), ion concentration may allow for a more robust estimation.

The processing engine may be configured to receive a plurality of parameters of the feed water, including but not limited to temperature (° C.), pH, carbon dioxide (CO₂) content, bicarbonate content, alkalinity, total dissolved solids (TDS, mg/L), conductivity (microsiemens per centimeter, μs/cm), turbidity, and/or total organic carbon (TOC, mg/L) of the feed water. Based on the pH and bicarbonate in the system, the total alkalinity and CO2 are calculated from theoretical formulas. In some embodiments, one or more of the aforementioned parameters may be determined by the processing engine using one or more of the input parameters. For example, the CO₂ content and/or total alkalinity may be determined based on the received pH and/or bicarbonate content.

One or more of the feed water parameters may be received from one or more sensors of the water filtration system and transmitted (e.g., via the filtration system itself and/or by a user via a user input device) to the processing engine. Parameters including but not limited to pH, CO₂, and total alkalinity may exhibit a unique relationship that can improve the estimation of scaling in the water filtration membranes.

At block 220, the method 200 may comprise determining one or more potential precipitates in the feed water based on the identification of ions present in the feed water and the concentration data for each of the ions present in the feed water. Determining one or more potential precipitates in the feed water may first comprise executing an algorithm to determine, based on the ions identified in the feed water, what compounds can be formed. The processing engine may be configured to evaluate several combinations of ions to determine the compounds that may be formed based on the identified ions. In some embodiments, the processing engine may be configured to access a configuration data storage (e.g., configuration data storage 120, which, as described herein, may comprise a dataset describing ions and compounds that may be present in feed water supplies) to retrieve a set of potential compounds. The processing engine may evaluate using this set of potential compounds and the set of ions identified in the feed water supply which compounds can be formed from the ions. For example, the dataset comprising potential compounds may, for each compound, include an associated listing of the ions that constitute the compound. The dataset may also include the name, formula, solubility constant (Kₛₚ), and product constant (Q) for each compound. The processing engine may be configured to automatically determine what compounds are formed by comparing the identified ions in the feed water to the theoretical ions for each compound. The processing engine may determine which compounds within the configuration data consist of ions identified in the feed water and may indicate these compounds accordingly for further processing.

In a non-limiting example, feed water data may comprise an identification of the ions barium (Ba²⁺), Calcium (Ca²⁺), Fluoride (F⁻), and Phosphate (PO₄³⁻). The processing engine may access a configuration data storage comprising information for compounds including but not limited to Barium Fluoride (BaF₂), Barium Bromide (BaBr₂), Fluorapatite (Ca₅(PO₄)₃F), and Hydroxyapatite (Ca₅(PO₄)₃OH). The data structure comprising an indication of the compound Barium Fluoride (BaF₂) may comprise an indication of the ions Barium (Ba²⁺) and Fluoride (F⁻). The data structure comprising an indication of the compound Barium Bromide (BaBr₂) may comprise an indication of the ions barium (Ba²⁺) and Bromine (Br⁻). The data structure comprising an indication of the compound Fluorapatite (Ca₅(PO₄)₃F) may comprise an indication of the ions may comprise an indication of the ions Calcium (Ca²⁺), Phosphate (PO₄³⁻), and Fluoride (F⁻). The data structure comprising an indication of the compound Hydroxyapatite (Ca₅(PO₄)₃OH) may comprise an indication of the ions may comprise an indication of the ions Calcium (Ca²⁺), Phosphate (PO₄³⁻), and Hydroxide (OH⁻). Each of the aforementioned compounds and their corresponding data structures may be stored in a single data structure or more than one data structure. The system may compare the ions present in the feed water to the ions stored in each of the compound data structures to automatically determine that the compounds Barium Fluoride (BaF₂) and Fluorapatite (Ca₅(PO₄)₃F) may be formed in a stream of the water filtration system, whereas the compounds Barium Bromide (BaBr₂) and Hydroxyapatite (Ca₅(PO₄)₃OH) may not be formed in the water filtration system. The pH measurement can be utilized to determine probability of existence of each compound.

In some embodiments, determining the one or more potential precipitates may be based on a solubility constant (Kₛₚ) and a product constant (Q) of one or more compounds formed by the identified ions in the feed water. For example, the processing engine may be configured to access the configuration data storage to retrieve a solubility constant (Kₛₚ) for each of the compounds determined above that may have been formed in the water filtration system. Continuing with the above example, the data structures for each of the aforementioned compounds may comprise a solubility constant. For example, Barium Fluoride (BaF₂) may have a Kₛₚ of 1.00×10⁻⁶, Barium Bromide (BaBr₂) may have a Kₛₚ of 1.72×10², Fluorapatite (Ca₅(PO₄)₃F) may have a Kₛₚ of 8.60×10⁻⁶¹, and Hydroxyapatite (Ca₅(PO₄)₃OH) may have a Kₛₚ of 1.00×10⁻³⁶.

The processing engine may be configured to determine the product constant (Q) for each of the compounds based on the concentration of the ions constituting the compound. As described herein, concentration data for each of the ions present in the feed water may be received by the processing engine, from the water filtration system itself (e.g., via one or more sensors and/or processors of the filtration system) and/or from a user via a user input device. Based on the concentration data of the ions in the feed water, the product constant (Q) for each compound that may have been formed in the water filtration system may be determined. The processing engine may be configured to store (e.g., in a RAM and/or an external data storage, such as configuration data storage 120) the determined product constants for each compound in the water filtration system for further processing.

Determining the one or more potential precipitates may comprise comparing the solubility constant to the product constant for each of the one or more compounds in the water filtration system. As is known to one of ordinary skill in the art, solubility constant Kₛₚ is representative of the solubility of the compound. A smaller Kₛₚ may indicate a compound that is more insoluble, and therefore may be more likely to precipitate and form scale on the membrane. On the other hand, a larger Kₛₚ may indicate a compound that is more soluble, and therefore may be less likely to precipitate and form scale on the membrane. However, solubility constant (Kₛₚ) on its own may be a poor indicator for estimating scale in the water filtration system. Therefore, the processing engine described herein may further consider the product constant (Q) of the compounds. As is known to one of ordinary skill in the art, product constant (Q) of a compound is based on the concentration of the ions constituting the compound. In some embodiments, comparing the solubility constant (Kₛₚ) with the product constant (Q) may comprise determining, for a given compound, which of the Kₛₚ and product constant Q is greater than the other. An ion product (Q) greater than the solubility constant (Kₛₚ) may indicate that a precipitate of the compound will likely be formed in the water filtration system, whereas a solubility constant (Kₛₚ) greater than the ion product (Q) may indicate that a precipitate of the compound likely will not form in the water filtration system. The processing engine may be configured to store (e.g., in a RAM and/or an external data storage, such as configuration data storage 120) the potential precipitates in the water filtration system, such as in the feed water stream, product stream, brine stream, etc.

In some embodiments, determining the one or more potential precipitates may comprise comparing characteristics of each of the potential precipitates to the other potential precipitates. For example, for each of the above-described potential precipitates whereby Q is greater than Kₛₚ, the determined difference in Q and Kₛₚ of a given potential precipitate may be compared to the determined Q and Kₛₚ differences of the remaining potential precipitates. The potential precipitates with the greatest difference between Q and Kₛₚ may be determined to be the most probable precipitates in the water filtration system.

In some embodiments, determining the one or more potential precipitates may comprise comparing characteristics of each of the potential precipitates to a threshold. For example, for each of the above-described potential precipitates whereby Q is greater than Kₛₚ, the difference in Q and Kₛₚ of each of the plurality of potential precipitates may be compared to a threshold Q and Kₛₚ difference. The potential precipitates that meet (or exceed) the threshold may be determined to be the most probable precipitates in the water filtration system. In some embodiments, the threshold may be pre-determined. In some embodiments, the threshold may be dynamically updated for each use case of the systems and methods described herein.

In some embodiments, determining the one or more potential precipitates may comprise using a regression model. The regression model may consider compound data and, in some embodiments, one or more parameters of the feed water. For example, the regression model may be configured to receive as input at least ion concentration data, solubility constant data, product constant data, and/or feed water pH data. In some embodiments, for each compound identified in the feed water data, the concentration data for the ions in the compound, the Kₛₚ of the compound, the Q of the compound, and the pH of the feed water may be provided to a regression model to determine whether the compound will form a precipitate. In some embodiments, the regression model may be configured to individually determine whether each compound of a plurality of compounds identified in a feed water supply can form a precipitate.

In some embodiments, the regression model may be configured to determine, at once, whether each compound of a plurality of compounds identified in a feed water supply can form a precipitate.

For example there could be an instance at this step that either (1) the magnitudes of the values are considered (e.g., in comparison to a threshold value) for determining most probable precipitates, or (2) the magnitudes of the different compounds are compared relative to one another to determine the most probable precipitates. Here the most probable scaling precipitates will be those that exhibit the greatest difference between Q and Ksp. Where Q>Ksp. Continuing with the above example, based on the formed compounds Barium Fluoride (BaF₂) and Fluorapatite (Ca₅(PO₄)₃F) and considering only the Kₛₚ, the processing engine may determine that Fluorapatite (Ca₅(PO₄)₃F), which has a Kₛₚ of 8.60×10⁻⁶¹, may be likely form a precipitate, whereas Barium Fluoride (BaF₂), which has a Kₛₚ of 1.00×10⁻⁶, may not be likely to form a precipitate. However, instances could exist in which the concentration of Barium ions (Ba²⁺) is very high, whereas the concentration of phosphate (PO₄³⁻) may be very low, which would contribute to the product constant determination of each of the compounds and therefore could lead to alternative determinations. For example, it may be determined that (1) both Barium Fluoride and Fluorapatite precipitates may be formed in a water stream of the water filtration system, or (2) a Barium Fluoride precipitate may be formed in the water stream of the water filtration system, whereas a Fluorapatite precipitate may not be formed in the water stream of the water filtration system. Therefore, it is important to consider the ion concentrations (e.g., via determining the product constant, Q) in combination with the solubility constant to determine potential precipitates that can be formed in the water filtration system (e.g., in a feed water stream, product stream, brine stream, etc. of the water filtration system).

In some embodiments, determining the one or more potential precipitates may be based on an induction time of each of the one or more compounds formed by the ions identified in the feed water. The induction time is the amount of time necessary for a reaction between ions to occur or initiate to form a compound. Here when the induction time is considered in relation to the product constant Q and solubility constant Ksp by experimental determination and is considered in the above-mentioned regression model for some commonly known scaling compounds. A compound with a long induction time may be less likely to form a precipitate than a compound with a short induction time.

The above-described regression model may be configured to consider induction time to determine whether a precipitate is likely to be formed from the compound. If the compound is in solution for a considerable time duration the compound will precipitate and this is known as the induction time. Continuing with the above example, in the instance each of the compounds Barium Fluoride (BaF₂) and Fluorapatite (Ca₅(PO₄)₃F) have a solubility constant (Kₛₚ) and product constant (Q) that lead to the determination that each of the compounds may form precipitates (i.e., a small Kₛₚ and/or large Q), the induction time of each of BaF₂ and Ca₅(PO₄)₃F may be considered to determine whether each of the compounds will precipitate.

For example, in one instance, Barium Fluoride may have a small induction time, whereas Fluorapatite may have a large induction time. The induction times of each of the compounds may be compared to known induction times for each of the compounds, and based on this comparison, the processing engine may determine that only a Barium Fluoride precipitate may be formed. As this is a non-limiting example, other instances could occur in which (1) both the compounds BaF₂ and Ca₅(PO₄)₃F have large induction times and therefore it is determined that neither compound forms a precipitate, (2) both the compounds BaF₂ and Ca₅(PO₄)₃F have small induction times and therefore it is determined that both compounds form a precipitate, or (3) BaF₂ has a small induction time whereas Ca₅(PO₄)₃F has a large induction time, and therefore only a BaF₂ compound forms a precipitate, or (4) both the compounds BaF₂ and Ca₅(PO₄)₃F have induction times that cause both compounds to form a precipitate in a stream of the water filtration system, but at different times (e.g., BaF₂ forms a precipitate before Ca₅(PO₄)₃F, or vice versa).

In some embodiments, the ions identified in the feed water that may form the potential precipitates may include but are not limited to calcium (Ca²⁺), carbonate (CO₃²⁻), hydroxide (OH⁻), phosphate (PO₄³⁻), sulfate (SO₄²⁻), Barium (Ba²⁺), Aluminum (Al³⁺), Oxide (O²⁻), Magnesium (Mg²⁺), Ammonium (NH⁴⁺), Fluoride (F−), Iron (III) (Fe³⁺), Hydrogen (H+), and/or Iron (II) (Fe2+) precipitates. At block 230, the method 200 may comprise determining a scaling potential of the water in the water filtration system. The scaling potential of the feed water may serve as a primary indicator of whether scaling will occur on the membrane(s) of a water filtration system, in pipes of the water filtration system, on pumps of the water filtration system, and in some embodiments, the extent (e.g., magnitude) to which scaling may occur on one or more of these aspects of the water filtration system. As described herein, the methods and systems may be configured to determine an amount of scaling in various streams of the water filtration system, such as the feed water stream, product stream, brine stream, and other waterways within the water filtration system.

Determining the scaling potential of the feed water may comprise using the one or more potential precipitates that have been previously determined (described above) in combination with one or more parameters of the feed water. As described above with reference to block 210, feed water data received by the processing engine may comprise a plurality of parameters of the feed water. Parameters may include but are not limited to temperature, pH, carbon dioxide (CO₂) content, bicarbonate content, alkalinity, total dissolved solids (TDS), conductivity, turbidity, and/or total organic carbon (TOC) of the feed water. One or more of the aforementioned parameters may be used to determine a Langelier Saturation Index (LSI) of the feed water. As is known by one of ordinary skill in the art, LSI is a common indicator of scalability because it approximates the degree of saturation of calcium carbonate (CO₃) in the system, which is known to be a common constituent of scale. Many pipelines in water filtration systems have calcium carbonate coatings, and undersaturated waters can rapidly remove this coating. LSI may be determined based on TDS or conductivity, calcium (Ca²⁺) concentration, bicarbonate (HCO³⁻) concentration, temperature, and pH. In some embodiments, determining the scaling potential of the water in the water filtration system may comprise comparing the determined LSI of the feed water to a predetermined threshold range. For example, an acceptable LSI threshold range may be between about −0.3 and 0.3. A negative LSI (below 0) may indicate that calcium carbonate is undersaturated, which can lead to irreversible corrosion of pipes in the water filtration system. A positive LSI (above 0) can indicate that the water is supersaturated, and some scale may form in the water filtration system. An LSI of 0 may indicate that the feed water is at saturation, so no calcium carbonate is removed and/or no scale may form.

Although, as mentioned above, calcium carbonate can be a common constituent of scale formed in water filtration systems, it is clearly not the only precipitate that can be formed from ions in the feed water. LSI does not account for any other ions in the feed water beyond calcium and bicarbonate. Accordingly, the systems and methods described herein consider additional parameters of potential precipitates to determine the scaling potential of feed water. For example, determining the scaling potential of the feed water may comprise using the determined LSI in combination with the solubility constants (Kₛₚ) and product constants (Q) of the potential precipitates in the water of the water filtration system. For example, for each of the determined precipitates in the water filtration system, the product constant (Q) and solubility constant (Kₛₚ) may be considered to determine the scaling potential.

In some embodiments, the system may determine an average difference in Q and Kₛₚ for all precipitates formed in the feed water. In some embodiments, the scaling potential may be based on a product of LSI and the average difference between Q and Kₛₚ. In some embodiments, the scaling potential may be based on a product of LSI and the logarithm of the average difference between Q and Kₛₚ. In some embodiments, LSI may only be considered in the instance the LSI value is within the predetermined threshold range.

For example, the scaling potential may consider LSI if the LSI is greater than 0.5, 1, 1.5, or 2. In the instance LSI is outside of the threshold range, the scaling potential may be based on the average difference between Q and Kₛₚ (or a variation, such as logarithm, thereof).Based on the LSI and characteristics of the determined precipitates in the feed water, the scaling potential of water stream(s) in the water filtration system can be determined. The scaling potential may be within a predetermined range (e.g., a scale) of values, otherwise referred to herein as a severity scale. For example, the severity scale may range from 1-10, 1-5, 1-100, 1-50, 1-20, 1-15, or another range of values. Each integer of the severity scale may be associated with, or assigned, one or more values based on the LSI and/or characteristics of the precipitates (e.g., Q and Kₛₚ).

In some embodiments, the scaling potential integers in the severity scale may be determined based on the average difference between product constant (Q) and the solubility constant (Kₛₚ) (or a logarithm thereof) and optionally the magnitude of the LSI for the precipitates formed from the feed water. In some embodiments, each integer within the severity scale may be assigned a range of values based on the average difference between Q and Kₛₚ for all of the precipitates and optionally the LSI of the feed water (e.g., in the instance LSI is greater than a threshold value, such as 1).

For example, the low end of the severity scale (e.g., 0, 1, etc.) may be associated with a low average difference between Q and Kₛₚ (or variations thereof, such as logarithm). In some embodiments, the low end of the severity scale may be associated with a low resultant product of the logarithm of the average difference between Q and Kₛₚ and LSI. The high end of the severity scale (e.g., 10, 15, 20, 100, dependent on the range of values selected) may be associated with a high average difference between Q and Kₛₚ (or variations thereof, such as logarithm). In some embodiments, the high end of the severity scale may be associated with a high resultant product of the logarithm of the average difference between Q and Kₛₚ and LSI.

A non-limiting example of values assigned to scaling potential values of a severity scale is provided below in Table 1. For simplicity, example ranges are provided for three scaling potential integers of the severity scale, 1, 5, and 10, however, it is to be understood that this example severity scale can include additional integer values (e.g., between 1 and 5 and between 5 and 10).

TABLE 1
Scaling Potential Values Example

| Scaling Potential | LSI × log(avg(Q − Kₛₚ)) |
|---|---|
| 10 (High) | >24 |
| 5 (Moderate) | 12-15 |
| 1 (Low) | 0-3 |

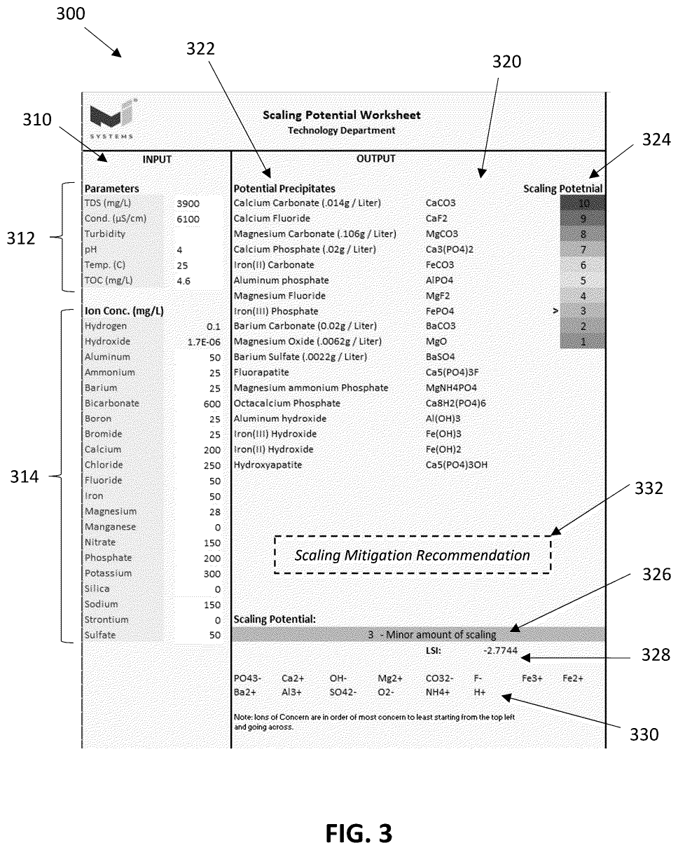

At block 240, the method 200 may comprise displaying output information to mitigate scaling of the water filtration system based on the determined scaling potential of the water. The displayed output information may comprise a representation of the scaling potential, such as the severity scale described above. In some embodiments, the displayed output information may alternatively or additionally comprise one or more of the LSI of the feed water, a representation of the ions present in the feed water, and/or a representation of the one or more potential precipitates in the water filtration system. Each of the example output information displayed to the user is described in greater detail herein with respect to FIG. 3.

For example, if LSI>1, If the product of LSI and the logarithmic value of the average difference between Ksp and Q for all potential precipitates is >24, that indicates a very severe amount of scaling.

if LSI>1, If the product of LSI and the logarithmic value of the average difference between Ksp and Q for all potential precipitates is between 12-15, that indicates a moderate amount of scaling.

if LSI>1, If the product of LSI and the logarithmic value of the average difference between Ksp and Q for all potential precipitates is between 0-3, that indicates little to no scaling.

If LSI<1, only the logarithmic value of the average difference between Ksp and Q for all precipitates is considered.

In some embodiments, the output information displayed may comprise a recommendation for mitigating scaling of the water filtration system. For example, the method may comprise generating, by the system, a recommendation to be displayed to the user to mitigate scaling and/or corrosion in the water filtration system. If the system predicts that the feed water may cause scaling of the membrane within the water filtration system, the recommendation may comprise an indication of one or more anti-scalant solutions to be added to the water filtration system, a dosage of the one or more anti-scalant solutions to be added to the water filtration system, and/or a flow rate for adding the one or more anti-scalant solutions to the water filtration system. In a similar nature, if the system predicts that the feed water may cause corrosion within the water filtration system, the recommendation may comprise an indication of one or more anti-corrosive solutions to be added to the water filtration system, a dosage of the one or more anti-corrosive solutions to be added to the water filtration system, and/or a flow rate for adding the one or more anti-corrosive solutions to the water filtration system. In some embodiments, the recommendation may comprise an indication of a time at which to next measure the feed water parameters and/or ion concentrations, for example, in the instance one or more of parameters/concentrations are not continuously measured and automatically analyzed by the system. For example, the output information displayed may inform the user to measure feed water parameters and/or ion concentrations again after a predetermined number of hours, days, weeks, months, etc. have passed.

In some embodiments, the method may comprise automatically causing, by the system, the water filtration system to dispense an anti-scalant and/or anti-corrosive solution based on the determined scaling potential. For example, based on the determined scaling potential, the system may be configured to automatically select an anti-scalant or anti-corrosive solution, a dosage of solution, and/or flow rate for dispensing the solution. In some embodiments, the system may be configured to determine a frequency (or cadence) at which to dispense the anti-scalant and/or anti-corrosive solution.

FIG. 3 illustrates a graphical user interface (GUI) 300 configured to receive inputs and display outputs for estimating scaling in a water filtration system. The GUI 300 may comprise an input region (e.g., a display window) 310 and an output region (e.g., a display window) 320.

With reference to the input region 310, the GUI 300 may comprise a parameter sub-region 312 and an ion sub-region 314. The parameter sub-region 312 may comprise one or more prompts configured to receive an indication of one or more feed water parameters from a user. In some embodiments, the parameter sub-region 312 may be configured to automatically populate based on one or more feed water parameters provided to the system by a communicatively coupled water filtration system. Parameters included in the parameter sub-region 312 may include but are not limited to total dissolved solids (TDS), conductivity, turbidity, pH, temperature, and total organic carbon (TOC). In some embodiments, the parameter sub-region 312 may be configured to receive an indication of one or more additional parameters not explicitly listed above. For example, one or more parameters may be pre-filled to the parameter sub-region 312 on the GUI 300, however, the GUI 300 may be configured to receive additional parameter inputs from the user.

The ion sub-region 314 may comprise one or more prompts configured to receive an indication of one or more ions in the feed water (and/or the concentration of the ion) from the user. In some embodiments, the ion sub-region 314 may be configured to automatically populate with ions and/or the concentration of the ions based on one or more measurements provided to the system by a communicatively coupled water filtration system. In some embodiments, the ion sub-region 314 may be pre-populated with a plurality of ions that may be identified in the feed water, and the user (and/or the water filtration system) may provide to the GUI 300 concentration data for each of the ions identified in the feed water. In some embodiments, the ion sub-region 314 may be configured to receive an indication of and/or information related to one or more ions in the feed water that are not already displayed within the sub-region, and the system may be configured to update the GUI 300 to display the information provided to the GUI 300.

The output region 320 may comprise one or more representations of system outputs, including but not limited to potential precipitates 322, a severity scale 324, a scaling potential 326, an LSI 328, potential ions 330, and/or a scaling mitigation recommendation 332.

The representation of potential precipitates 322 may comprise a listing (or table) of each of the potential precipitates determined by the system to be formed in the water filtration system. The listing of potential precipitates 322 may comprise the chemical names and/or chemical formulas for each of the precipitates. In some embodiments, the precipitates in the listing may be displayed in a randomized or particular order. For example, the precipitates may be ranked based on prevalence (e.g., concentration) in the feed water, risk, etc. In some embodiments, the precipitates may be ranked from highest potential to scale the water filtration system to lowest potential to scale the system.

The severity scale 324 may comprise a visual representation of a numerical scale. In some embodiments, the severity scale 324 may be color-coordinated. For example, as shown in FIG. 3, the severity scale 324 may range from 1 to 10. As a low scaling potential (e.g., a 1) is preferable, a value of 1 on the severity scale may be associated with a positively connoted color, such as green. On the other hand, a high scaling potential (e.g., a 10), which is less preferable, may be associated with a negatively connoted color, such as red. Between the two ends on the scale, a corresponding spectrum of colors (e.g., ranging between shades of green and red, as well as yellow and orange) may be associated with the intermediate scaling potential values. The severity scale 324 may comprise a visual marking, such as an arrow, shaped outline, or other marking that indicates the scaling potential determined by the system. For example, in the GUI 300 shown in FIG. 3, a scaling potential of “3” is indicated in the severity scale 324.

This scaling potential may additionally or alternatively be indicated elsewhere in the GUI 300, such as shown in FIG. 3 by the representation of scaling potential 326. In some embodiments, scaling potential 326 may comprise a numerical representation of the scaling potential (e.g., a “3” for the example shown in FIG. 3). In some embodiments, the scaling potential 326 may additionally or alternatively comprise a textual representation, such as a word, phrase, and/or sentence associated with the determined scaling potential. For example, as shown in FIG. 3, the scaling potential 326 may comprise the phrase “minor amount of scaling.” In some embodiments, the representation of the scaling potential 326 in the GUI 300 may be colored, for example, to correspond with the coloring of the severity scale 324. Table 2 provided below demonstrates example textual representations for each scaling potential value in a severity scale with integers in the range of 1-10.

TABLE 2
Severity Scale Example

| | |
|---|---|
| 10 | Very severe amount of scaling |
| 9 | Severe amount of scaling |
| 8 | Intensive amount of scaling |
| 7 | Considerable amount of scaling |
| 6 | Moderate amount of scaling |
| 5 | Slight amount of scaling |
| 4 | Minimal amount of scaling |
| 3 | Minor amount of scaling |
| 2 | Little to no scaling |
| 1 | Very unlikely to have scaling |

The representation of the LSI 328 may comprise a numerical representation of the LSI determined by the system, as shown. For example, in the GUI 300 of FIG. 3, the LSI 328 is −2.7744.

The representation of potential ions 330 may comprise a listing (or table) of each of the ions identified in the feed water. The listing of potential ions 330 may comprise the chemical names and/or chemical formulas for each of the ions. In some embodiments, the ions in the listing may be displayed in a randomized or particular order. For example, the ions may be ranked based on prevalence (e.g., concentration) in the feed water, risk, etc. In some embodiments, the ions may be ordered from ion of most concern to ion of least concern.

As described herein, in some embodiments, the output region 320 of the GUI 300 may comprise a scaling mitigation recommendation 332. The recommendation 332 may comprise a textual and/or illustrative representation of one or more mitigation strategies for the user to mitigate scaling and/or corrosion in the water filtration system. As described herein, the recommendation may comprise an indication of an anti-scalant and/or anti-corrosive solution to be added to the water filtration system, a dosage of solution, and/or a flow rate of the solution.

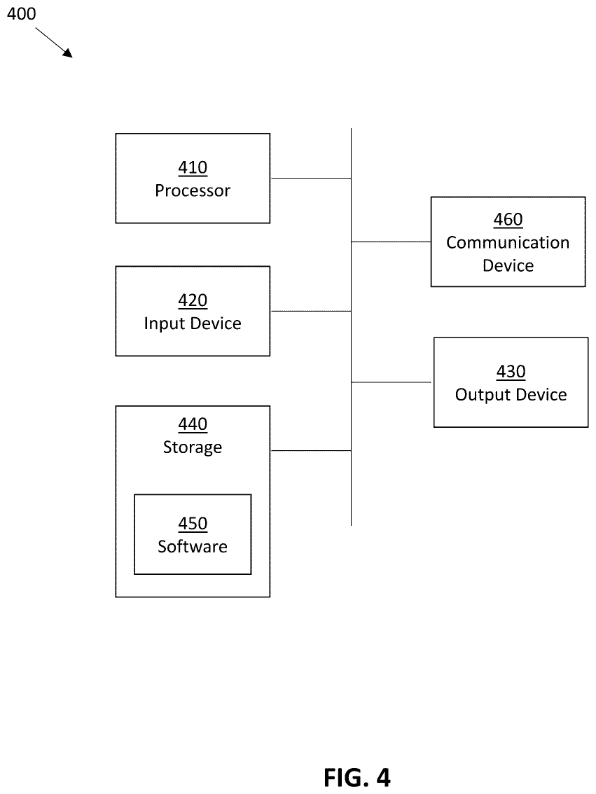

FIG. 4 illustrates a computer, in accordance with some embodiments. Computer 400 can comprise any one or more components of system 100 described above with reference to FIG. 1. In some embodiments, computer 400 may be configured to execute a method for automatically generating computer programs, such as computer programs for estimating the amount of scaling in a water filtration system (described herein with respect to method 300). In some embodiments, computer 400 may be configured to execute any of the other techniques discussed herein, alone and/or in combination with one another and/or with method 300.

Computer 400 can be a host computer connected to a network. Computer 400 can be a client computer or a server. As shown in FIG. 4, computer 400 can be any suitable type of microprocessor-based device, such as a personal computer; workstation; server; or handheld computing device, such as a phone or tablet. The computer can include, for example, one or more of processor 410, input device 420, output device 430, storage 440, and communication device 460.

Input device 420 can be any suitable device that provides input, such as a touch screen or monitor, keyboard, mouse, or voice-recognition device. Output device 430 can be any suitable device that provides output, such as a touch screen, monitor, printer, disk drive, or speaker. Storage 440 can be any suitable device that provides storage, such as an electrical, magnetic, or optical memory, including a RAM, cache, hard drive, CD-ROM drive, tape drive, or removable storage disk. Communication device 460 can include any suitable device capable of transmitting and receiving signals over a network, such as a network interface chip or card. The components of the computer can be connected in any suitable manner, such as via a physical bus or wirelessly. Storage 440 can be a non-transitory computer-readable storage medium comprising one or more programs, which, when executed by one or more processors, such as processor 410, cause the one or more processors to execute methods described herein, such as all or part of method 300.

Software 450, which can be stored in storage 440 and executed by processor 410, can include, for example, the programming that embodies the functionality of the present disclosure (e.g., as embodied in the systems, computers, servers, and/or devices as described above). In some embodiments, software 450 can be implemented and executed on a combination of servers such as application servers and database servers.

Software 450 can also be stored and/or transported within any computer-readable storage medium for use by or in connection with an instruction execution system, apparatus, or device, such as those described above, that can fetch and execute instructions associated with the software from the instruction execution system, apparatus, or device. In the context of this disclosure, a computer-readable storage medium can be any medium, such as storage 440, that can contain or store programming for use by or in connection with an instruction execution system, apparatus, or device.

Software 450 can also be propagated within any transport medium for use by or in connection with an instruction execution system, apparatus, or device, such as those described above, that can fetch and execute instructions associated with the software from the instruction execution system, apparatus, or device. In the context of this disclosure, a transport medium can be any medium that can communicate, propagate, or transport programming for use by or in connection with an instruction execution system, apparatus, or device. The transport-readable medium can include, but is not limited to, an electronic, magnetic, optical, electromagnetic, or infrared wired or wireless propagation medium.

Computer 400 may be connected to a network, which can be any suitable type of interconnected communication system. The network can implement any suitable communications protocol and can be secured by any suitable security protocol. The network can comprise network links of any suitable arrangement that can implement the transmission and reception of network signals, such as wireless network connections, T1 or T3 lines, cable networks, DSL, or telephone lines. Computer 400 may be composed of components which are interconnected across a network, such as a distributed system. Computer 400 may be organized into any suitable topology, such as a star topology, a recursively defined topology, a mesh topology, a ring topology, or an ad-hoc topology.

Computer 400 can implement any operating system suitable for operating on the network. Software 450 can be written in any suitable programming language, such as C, C++, Java, or Python. In various embodiments, application software embodying the functionality of the present disclosure can be deployed in different configurations, such as in a client/server arrangement or through a Web browser as a Web-based application or Web service, for example.

The foregoing description, for the purpose of explanation, has been described with reference to specific embodiments. However, the illustrative discussions above are not intended to be exhaustive or to limit the invention to the precise forms disclosed. Many modifications and variations are possible in view of the above teachings. The embodiments were chosen and described in order to best explain the principles of the techniques and their practical applications. Others skilled in the art are thereby enabled to best utilize the techniques and various embodiments with various modifications as are suited to the particular use contemplated.

Although the disclosure and examples have been fully described with reference to the accompanying figures, it is to be noted that various changes and modifications will become apparent to those skilled in the art. Such changes and modifications are to be understood as being included within the scope of the disclosure and examples as defined by the claims. Finally, the entire disclosure of the patents and publications referred to in this application are hereby incorporated herein by reference.

What is claimed is:

1. A method for estimating the amount scaling in a water filtration system, comprising:
receiving feed water data comprising at least an identification of ions present in the feed water, concentration data for each of the ions present in the feed water, and a plurality of parameters of the feed water;
determining one or more potential precipitates in the feed water based on the identification of ions present in the feed water and the concentration data for each of the ions present in the feed water; and
based at least one the one or more potential precipitates and the plurality of parameters of the feed water, determining the scaling potential of the feed water to estimate the amount of scaling in the water filtration system and wherein ion concentration is directly related to scale formation on membrane surfaces.

2. The method of claim 1, wherein the plurality of parameters of the feed water comprises temperature, pH, CO₂ content, and/or alkalinity of the feed water.

3. The method of claim 1, wherein determining the one or more potential precipitates is based on a solubility constant (Kₛₚ) and a product constant (Q) of one or more compounds formed by the identified ions in the feed water.

4. The method of claim 3, wherein determining the one or more potential precipitates comprises comparing the solubility constant to the product constant for each of the one or more compounds in the feed water.

5. The method of claim 4, wherein determining the one or more potential precipitates is based on an induction time of each of the one or more compounds formed by the identified ions in the feed water.

6. The method of claim 5, wherein determining the one or more potential precipitates comprises using a regression model that is configured to receive as input concentration data for each of said ions present in said feed water, solubility constant data, product constant data, and/or feed water pH data, and to provide as output whether precipitates will form and if one or more potential precipitates will precipitate out of solution.

7. The method of claim 6, wherein the one or more potential precipitates comprises calcium, carbonate, hydroxide, phosphate, and/or sulfate precipitates.

8. The method of claim 7, wherein determining the scaling potential of the membrane comprises comparing a determined Langelier Saturation Index (LSI) of the feed water to a predetermined threshold range wherein said scaling potential utilizes a model that reviews all common scaling compounds and feed water composition to determine if ions are present in said feed water wherein an absence of ions in said scaling compound leads to no precipitate and wherein a product constant (Q) is calculated and compared it a solubility constant (Ksp) of said product such that if Q>Ksp there is potential for scaling to occur and wherein pH and calcium, carbonate content determines said Langelier Saturation Index (LSI) and if LSI>1, said LSI and said Ksp and said ion concentration determine a degree of scaling and wherein if LSI<1, said Ksp and ion concentration are combined to determine a degree of scaling.

9. The method of claim 8, comprising displaying output information to mitigate membrane scaling based on the determined scaling potential of the water.

10. The method of claim 9, wherein the displayed output information comprises a representation of the scaling potential.

11. The method of claim 10, wherein the representation of the scaling potential comprises a severity scale.

12. The method of claim 11, wherein the displayed output information comprises the Langelier Saturation Index (LSI) of the feed water.

13. The method of claim 12, wherein the displayed output information comprises a representation of the ions present in the feed water and/or a representation of the one or more potential precipitates in the feed water.

14. The method of claim 13, wherein the output information comprises one or more anti-scalant solutions to be added to the water filtration system, a dosage of the one or more anti-scalant solutions to be added to the water filtration system, and/or a flow rate for adding the one or more anti-scalant solutions to the water filtration system.

15. The method of claim 14, wherein the membrane in the water filtration system utilizes electrodialysis, reverse osmosis, nanofiltration, and/or ultrafiltration.

16. A system for estimating scaling of a membrane in a water filtration system, the system comprising one or more processors configured to cause the system to:
receive feed water data comprising at least an identification of ions present in the feed water, concentration data for each of the ions present in the feed water, and a plurality of parameters of the feed water;
determine one or more potential precipitates in the feed water based on the identification of ions present in the feed water and the concentration data for each of the ions present in the feed water; and
based at least one the one or more potential precipitates and the plurality of parameters of the feed water, determine a scaling potential of the feed water to estimate scaling in the water filtration system.

17. The system of claim 16, wherein the plurality of parameters of the feed water comprises temperature, pH, CO2 content, and/or alkalinity of the feed water.

18. The system of claim 17, wherein determining the one or more potential precipitates is based on a solubility constant (Kₛₚ) and a product constant (Q) of one or more compounds formed by the identified ions in the feed water and wherein determining the one or more potential precipitates comprises comparing the solubility constant to the product constant for each of the one or more compounds in the feed water.

19. The system of claim 18, wherein determining the one or more potential precipitates is based on an induction time of each of the one or more compounds formed by the identified ions in the feed water.

20. The system of claim 19, wherein determining the one or more potential precipitates comprises using a regression model configured to receive as input the concentration data for each of the ions present in the feed water, solubility constant data, product constant data, and/or feed water pH data, and to provide as output the one or more potential precipitates.

21. The system of claim 20, wherein the one or more potential precipitates comprises calcium, carbonate, hydroxide, phosphate, and/or sulfate precipitates.

22. The system of claim 21, wherein determining the scaling potential of the membrane comprises comparing a determined Langelier Saturation Index (LSI) of the feed water to a predetermined threshold range.

23. The system of claim 22, comprising displaying output information to mitigate membrane scaling based on the determined scaling potential of the water.

24. The system of claim 23 wherein the displayed output information comprises a representation of the scaling potential.

25. The system of claim 24, wherein the representation of the scaling potential comprises a severity scale.

26. The system of claim 25, wherein the displayed output information comprises the Langelier Saturation Index (LSI) of the feed water.

27. The system of claim 26, wherein the displayed output information comprises a representation of the ions present in the feed water and/or a representation of the one or more potential precipitates in the feed water.

28. The system of claim 27, wherein the output information comprises one or more anti-scalant solutions to be added to the water filtration system, a dosage of the one or more anti-scalant solutions to be added to the water filtration system, and/or a flow rate for adding the one or more anti-scalant solutions to the water filtration system.

29. The system of claim 28, wherein the membrane in the water filtration system utilizes electrodialysis, reverse osmosis, nanofiltration, and/or ultrafiltration.

30. A non-transitory computer-readable storage medium storing one or more programs for estimating the amount of scaling in a water filtration system, the programs for execution by one or more processors of an electronic device that when executed by the device, cause the device to:
receive feed water data comprising at least an identification of ions present in the feed water, concentration data for each of the ions present in the feed water, and a plurality of parameters of the feed water;
determine one or more potential precipitates in the feed water based on the identification of ions present in the feed water and the concentration data for each of the ions present in the feed water; and
based at least one the one or more potential precipitates and the plurality of parameters of the feed water, determine a scaling potential of the feed water to estimate the amount of scaling in the water filtration system.
